(19) United States
(12) Patent Application Publication
(10) Pub. No.: US 2011/0276051 A1
(43) Pub. Date: Nov. 10, 2011

(54) TETHER AND APPARATUS FOR PERFORMING A BONE RESECTION AND METHOD OF USE

(75) Inventors: David BLAKEMORE, Warsaw, IN (US); Eric M. LUCAS, Columbia City, IN (US); Keith Pennington, Warsaw, IN (US)

(73) Assignee: VOT, LLC, Warsaw, IN (US)

(21) Appl. No.: 13/044,006

(22) Filed: Mar. 9, 2011

Related U.S. Application Data
(60) Provisional application No. 61/312,096, filed on Mar. 9, 2010.
(60) Provisional application No. 61/312,093, filed on Mar. 9, 2010.

(51) Int. Cl. A61B 17/56

(52) U.S. Cl. 606/87

(57) ABSTRACT

A resection tether that includes an elongated member having a medial end and a lateral end. The lateral end is configured for attachment to a cutting device and the medial end is configured for attachment to a reference locator positioned in a bone. When attached at both ends, the resection tether controls the movement of the cutting device relative to the reference locator. Further disclosed is an apparatus that includes a tether having an elongated member that includes a medial end and a lateral end. The lateral end is configured for attachment to a bone cutting device and the medial end configured to attach a position on a bone. The apparatus includes a bone cutting device and a reference locator that has been positioned within a bone. Also disclosed are methods for using a tether to perform a bone resection and for creating an inlay bone resection.

### CROSS-REFERENCE TO RELATED APPLICATIONS

This application is entitled to the benefit of pending U.S. Provisional Patent Application Ser. No. 61/312,096 filed on Mar. 9, 2010 and pending U.S. Provisional Patent Application Ser. No. 61/312,093 filed on Mar. 9, 2010, the disclosures of which are included by reference herein in their entirety.

### TECHNICAL FIELD

This invention relates generally to the field of surgical instruments, and more specifically to the field of artificial knee surgical instruments.

### BACKGROUND OF THE INVENTION

Surgical procedures often require the removal of a section or specific area of bone in order to accommodate an implant and bone cement. In these types of surgical procedures it is often necessary to remove only a focal area of the bone without harming or damaging the remaining bone stock. Any damage to the surrounding bone may compromise or weaken the remaining bone and subsequent bone-implant interface resulting in implant failure. However, it is often difficult to visualize the precise area of the bone to be resected and bone cutting tools can be difficult to control. Therefore, it is a challenge to precisely remove the correct amount and location of bone necessary for the implantation of a medical device.

Accordingly, it is recognized that there is a need for a device and apparatus which allows a surgeon to easily and rapidly remove or resect a section of bone in a controlled manner where the surgeon's view of the surgical site may be limited.

### SUMMARY OF THE INVENTION

Advancement of the state of surgical instrumentation that are controlled when bone is cut, and more specifically, when a bone resection is performed for the implantation of a medical device is believed to be desirable. One example of an embodiment of the invention that satisfies the need for improvements to a controlled surgical instrument used to perform a bone resection includes an elongated member with medial and lateral ends that are both configured to facilitate the attachment to a reference locator and a cutting device. The aspects of the invention disclosed herein use a resection tether that, among other things, controls the movement of the cutting device during the bone resection process and maintains the desired distance between the reference locator and the cutting device during the operation of the cutting device.

The present invention provides, in one aspect, a resection tether having an elongated member with a medial end and a lateral end and a longitudinal axis that extends between the two ends. The lateral end is configured for attachment to a cutting device and the medial end is configured for attachment to a reference locator in a bone. When the two ends are attached, the resection tether controls the movement of the cutting device relative to the reference locator.

The present invention provides, in another aspect, an apparatus for performing a bone resection that has a tether that includes an elongated member having a medial end and a lateral end with a longitudinal axis extending between the ends. The lateral end is configured to attach to a bone cutting device and the medial end is configured to attach to a position on a bone. The apparatus also includes a bone cutting device and a reference locator positioned within a bone. The tether functions to connect the bone cutting device to the reference locator to control movement between the bone cutting device and the reference locator when the operator is performing a bone resection.

Another embodiment of the invention is a method of using a tether to perform a bone resection including the step of selecting a tether with a certain length. The method may also include the step of attaching the tether to a cutting device. The method may further include the step of attaching the tether to a reference locator. An additional step of operating the cutting device to resect the bone may be undertaken in the method.

A still further embodiment of the invention is a method of creating an inlay bone resection including the step of selecting a cutting device. The method may also have the step of selecting a tether that is adapted to control the movement of the cutting device. The method may have the further step of attaching the tether to the cutting device. The step of attaching the tether to a reference locator may also be included in the method. The method may further include the step of operating the cutting device to create an inlay bone resection.

Other additional features, benefits, and advantages of the present invention will become apparent from the following drawings and descriptions of the invention. Other embodiments of the invention are described in detail herein and are considered a part of the claimed invention.

### BRIEF DESCRIPTION OF THE DRAWINGS

The subject matter which is regarded as the invention is particularly pointed out and distinctly claimed at the end of the specification. The foregoing and other objects, features, and advantages of the invention are apparent from the following detailed description taken in conjunction with the accompanying drawings in which:

FIG. 1 is a perspective view of one embodiment of a resection tether, in accordance with an aspect of the invention;

FIG. 2 is a top view of the resection tether of FIG. 1, in accordance with an aspect of the invention;

FIG. 3 is a top view of an alternative embodiment of the resection tether of FIG. 1, in accordance with an aspect of the invention;

FIG. 4 is a top view of the resection tether of FIG. 1, attached to a cutting device, in accordance with an aspect of the invention;

FIG. 5 is a bottom view of the resection tether of FIG. 1, attached to a cutting device, in accordance with an aspect of the invention;

FIG. 6 is a bottom view of an alternative embodiment of the resection tether of FIG. 1, in accordance with an aspect of the invention;

FIG. 7A is a top view of an alternative embodiment of the resection tether of FIG. 1, in accordance with an aspect of the invention;

FIG. 7B is a top view of an alternative embodiment of the resection tether of FIG. 1, in accordance with an aspect of the invention;

FIG. 7C is a top view of an alternative embodiment of the resection tether of FIG. 1, in accordance with an aspect of the invention;

FIG. 8 is a top view of an alternative embodiment of the resection tether of FIG. 1, in accordance with an aspect of the invention;

FIG. 9 is a perspective view of the apparatus including the resection tether of FIG. 1 where the resection tether has been attached to a cutting device and a reference locator positioned within a bone, in accordance with an aspect of the invention;

FIG. 10 is an exploded view of the apparatus of FIG. 9, in accordance with an aspect of the invention;

FIG. 11 is the diagram showing the method of performing a bone resection, in accordance with an aspect of the invention; and

FIG. 12 is a diagram showing the method of creating an inlay bone resection.

### DETAILED DESCRIPTION FOR CARRYING OUT THE INVENTION

Generally stated, disclosed herein is a resection tether and an apparatus for performing a surgical resection. Further, disclosed herein is a method of using the resection tether to make a bone resection. Also disclosed is a method of creating an inlay bone resection. Any alterations and further modifications in the described embodiments, and any further applications of the principles of the invention as described herein are contemplated as would normally occur to one skilled in the art to which the resection tether invention relates.

In this detailed description and the following claims, the words proximal, distal, anterior, posterior, medial, lateral, superior and inferior are defined by their standard usage for indicating a particular part of a surgical instrument or surgical opening according to the relative disposition of the surgical instrument, surgical opening or directional terms of reference. For example, “proximal” means the portion of the surgical instrument positioned nearest the torso while “distal” indicates the part of the surgical instrument farthest from the torso. As for directional terms, “anterior” is a direction towards the front side of the body, “posterior” means a direction towards the back of the body, “medial” means towards the midline of the body, “lateral” is a direction towards the sides or away from the midline of the body, “superior” means a direction above, and “inferior” means a direction below another object or structure.

As used herein, the terms “tether” and “resection tether” may be used interchangeably as they essentially describe the same type of surgical instrument. In addition, the terms “cutting device” and “cutting tool” are contemplated to include any surgical instrument that is capable of cutting, resecting, burring, or removing bone or other tissue and the terms “apparatus”, “apparatus for performing a resection” and “apparatus for performing a resection in part or in whole” are used interchangeably to describe the same type of surgical device.

Generally stated, disclosed herein is a tether for use in surgical procedures. The tether shown herein is intended for example purposes only, as many alterations would occur to one skilled in the art, and are contemplated as a part of the invention. The tether generally is configured to limit the movement of a cutting device relative to a reference location, and includes a means for attaching to the cutting device, and a means for attaching to a fixed reference locator placed in a bone.

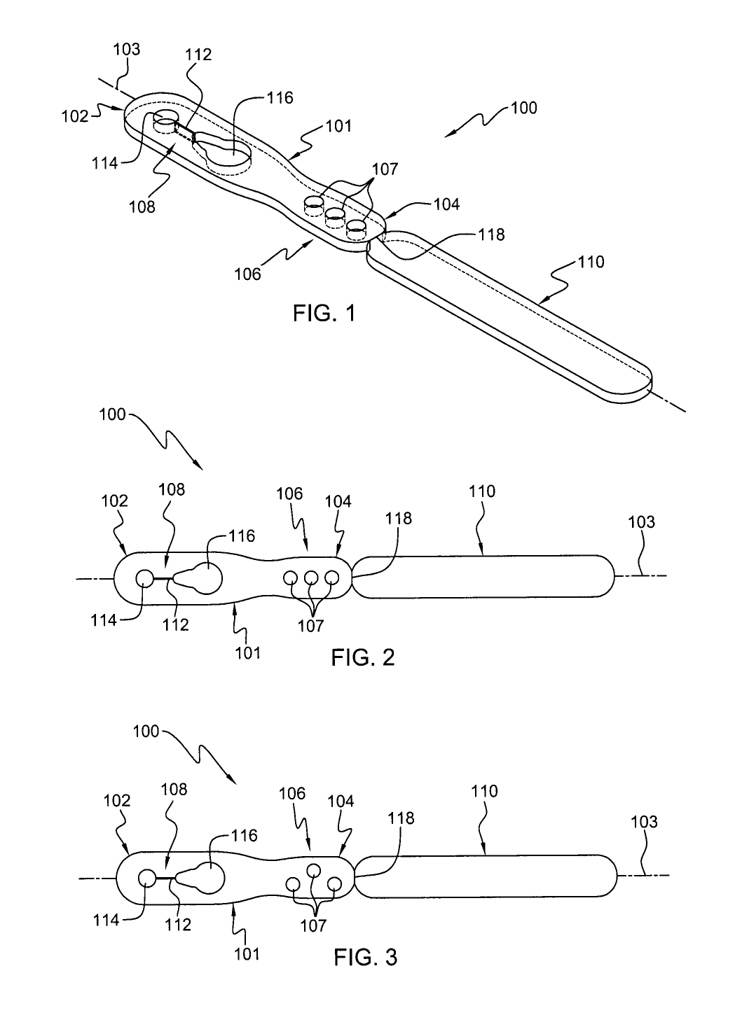

Referring now to FIG. 1, resection tether 100 has an elongated member 101 with a lateral end 102 and a medial end 104. A longitudinal axis 103 extends between lateral end 102 and medial end 104. A means for attaching to a reference locator in the bone 106 is seen at medial end 104. A means for attaching to a cutting device 108 may be formed on lateral end 102. A handle 110 may be attached to medial end 104.

FIG. 2 is a top view of resection tether 100 that shows means for attaching to a reference locator in a bone 106 as a plurality of holes 107 formed proximate to medial end 102. It is contemplated that means for attaching to a reference locator 106 may be one or more apertures through which a locator, such as a Kirschner wire or K-wire, pin, screw or other medical fastening device may be placed. Each of holes 107 have a center point that lie along the longitudinal axis 103. Alternatively, as shown in FIG. 3 the center point for holes 107 may be offset from longitudinal axis 103. Holes 107 are spaced a set distance from each other to correspond with the various sized implants that will be used to sit within the resected bone. For example, hole 107 that is located closest to the mid-point of elongated member 101 may be used for the smallest sized implant with hole 107 positioned closest to medial end 104 may be used to prepare the bone for the larger implant.

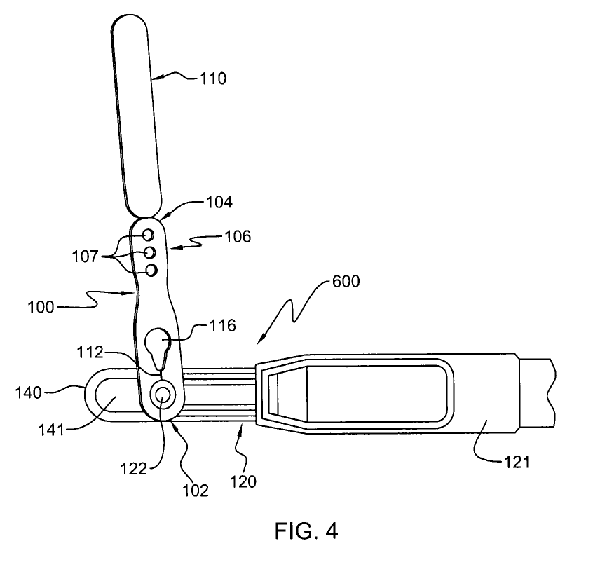

FIGS. 1 and 2 also show the means for attaching to a cutting device 108, which is positioned proximate to lateral end 102. In the embodiments shown, means for attaching to a cutting device 108 is an enlarged opening shaped for example purposes similar to a teardrop 116 with a through slit 112 extending in a lateral direction and intersecting with a second opening 114. Slit 112 is configured to facilitate the insertion and securement of a button, screw head, nail head, disk, post, clasp or other projection which may extend from a portion of the cutting device 120 or other surgical instrument (see FIG. 4). It is understood that when the button or other projection has been inserted through slit 112, tether 100 is free to rotate while the button or other projection rests within second opening 114. In an alternative embodiment, it is contemplated that the button or other projection may also rest within enlarged opening 116. In the embodiments shown in FIGS. 1 and 2, second opening 114 is shown as circular. However, it is contemplated that second opening 114 may be square, oval, circular, triangular, rectangular, or polygonal. In an alternative embodiment, it is contemplated that second opening 114 or enlarged opening 116 may be omitted from resection tether 100, or alternatively positioned in the location of slit 112.

FIG. 4 exhibits a top view of the apparatus 600 that is used to perform a bone resection. Apparatus 600 includes resection tether 100 that has been attached to a cutting device 120. A button 122 that is fixed to cutting device 120 is shown projecting through tether 100 via slit 112 and seats within second opening 114. Also shown are other components of cutting device 120, including a handle 121, a bur guard 140 having a top surface 141 on which button 122 is disposed. Typically, bur guard 140 is configured to protect surrounding tissue from being accidentally damaged by the cutting bur when operated.

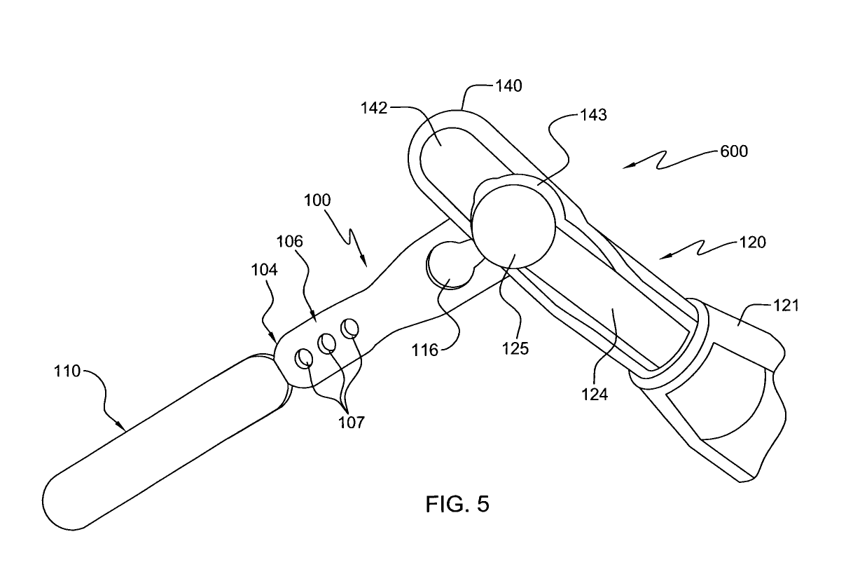

FIG. 5 is a bottom view of apparatus 600 with cutting device 120 attached to tether 100. Projecting from handle 121 is bur 124 with the cutting head 125 being positioned distally in a depression 143 disposed on the bottom surface 142 of guard 140. Handle 110 and medial end 104 are shown to overhang bur guard 140 when tether 100 is secured to guard 140.

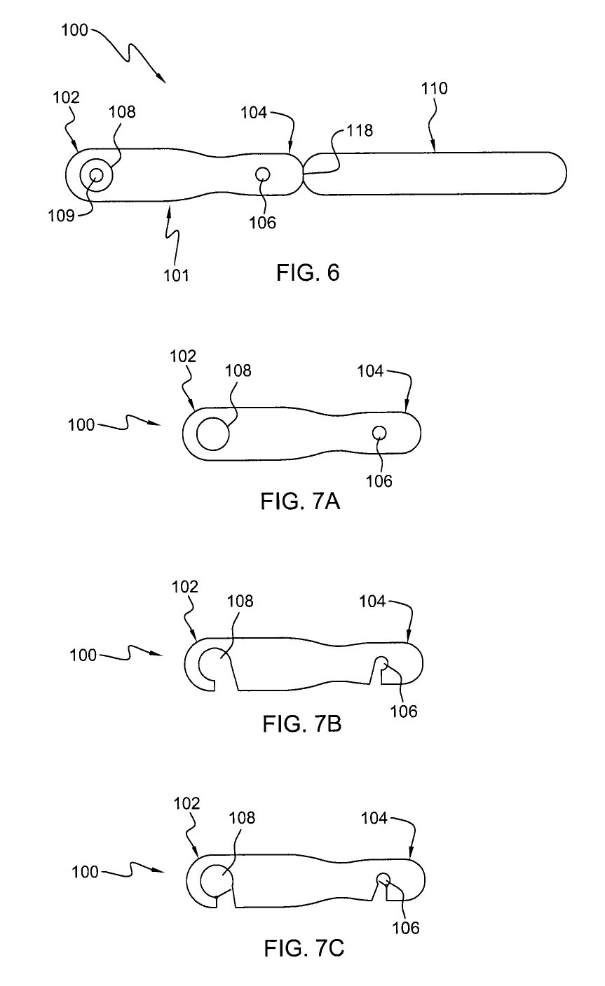

An alternative embodiment of tether 100 is shown in FIG. 6, with means for attaching to a cutting device 108 being a snap fit mechanism 109. Snap fit mechanism 109 would include a male portion on top surface 141 of bur guard 140 with a female portion positioned proximate to lateral end 102. In operation, tether 100 would be circumferentially moveably secured to top surface 141 of bur guard 140. The orientation of the male and female portions of snap fit mechanism 108 could also be reversed.

FIGS. 1 and 2 also show handle 110 as being attached to medial end 104. Handle 110 may be either fixedly attached or removably attached to medial end 104. For the example of handle 110 being removably attached, it is understood that handle 110 may be detached using a cutting instrument such as surgical scissors, a scalpel or other sharp surgical instrument. Tether 100 may be perforated, folded or otherwise structurally weakened at line 118 as shown in FIGS. 1-3 and 6, such that when a sufficient amount of force is applied, handle 110 will break away from medial end 104.

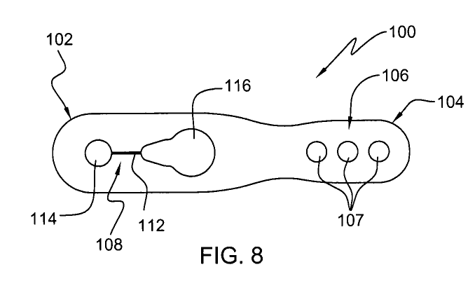

FIG. 8 further shows an alternative embodiment of tether 100. Specifically, tether 100 being fabricated without a handle 110. For this construct, tether 100 would only include medial end 104 and lateral end 102 with the same means for attaching to cutting device and reference locator (106, 108) that have already been described above and for brevity will not be repeated here.

As described above, tether 100 may utilize alternative means for attaching to the cutting device 108 and the reference locator 106 than the various configured fixed openings. As shown in FIGS. 7A-7C, several alternative embodiments of resection tether 100 are shown with such means. Specifically, FIG. 7A is a top view of an embodiment of resection tether 100 that exhibits a loop of material at medial end 104 and lateral end 102. It is understood that the loop may be placed around a screw, button, knob, hook or other projection attached to top surface 141 of guard 140. Further, the means for attaching to a reference locator 106 may also be a loop. The loop may be placed around or over a wire, screw, pin, bolt or other common surgical fastening device that has been or will be placed in a certain reference location in a bone. The loops are configured to allow for tightening or loosening around the attachment sites.

FIG. 7B shows a further alternative embodiment of tether 100 where means for attaching to a reference locator 106 may be a hook that is placed around a wire, pin, screw, bolt, or other common surgical fastening device that has been or will be placed in a reference location in a bone. Also shown is the means for attaching to a cutting device 108 using a hook. The hook may be inserted into an opening, or around a button, screw, knob, or other projections extending from top surface 141 of guard 140.

As seen in FIG. 7C, tether 100 may include a means for attaching to a reference locator 106 that is configured as a biased or spring clasp. The clasp would be placed or attached around a wire, pin, screw, bolt, or other common medical fastening device. Further, a clasp may also be used for the means for attaching to a cutting device 108. The clasp would be secured through an opening or on or around a button, screw, nail, bolt, or other projection disposed on top surface 141 of guard 140.

For all embodiments shown in FIGS. 7A-7C, the length of resection tether 100 may be fixed or adjustable. In the embodiment where resection tether 100 is of a fixed length, a kit may be comprised of multiple resection tethers 100 of different lengths, allowing a surgeon user to select a tether 100 of appropriate length to match the corresponding implant. Where the length of resection tether 100 is adjustable, it is contemplated that resection tether 100 may be extended or shortened to predetermined lengths. In another embodiment, where the length of tether 100 is adjustable, it is contemplated that tether 100 may be extended or shortened to any desired length via a variable mechanism.

It should be noted that the above described alternative means for attaching to a cutting device 108 may be combined with the described various means for attaching to a reference locator 106. For example, means for attaching to a cutting device 108 may be a hook, while means for attaching to a reference locator 106 may be a loop and vice-a-versa, for the same tether 100.

Various materials may be used to fabricate tether 100. Typically, a flexible, biocompatible, autoclaveable material such as nylon, acetal copolymers, homo polymers or PEEK may be used. Alternative embodiments of tether 100 may be comprised of an inflexible biocompatible, autoclavable material, such as hard plastic. Additionally, flexible biocompatible materials that may be sterilized through methods that do not involve an autoclave may also be used. Tether 100 may have a hybrid construct, wherein the tether is partially comprised of a flexible material and partially comprised of an inflexible material for certain surgical procedures.

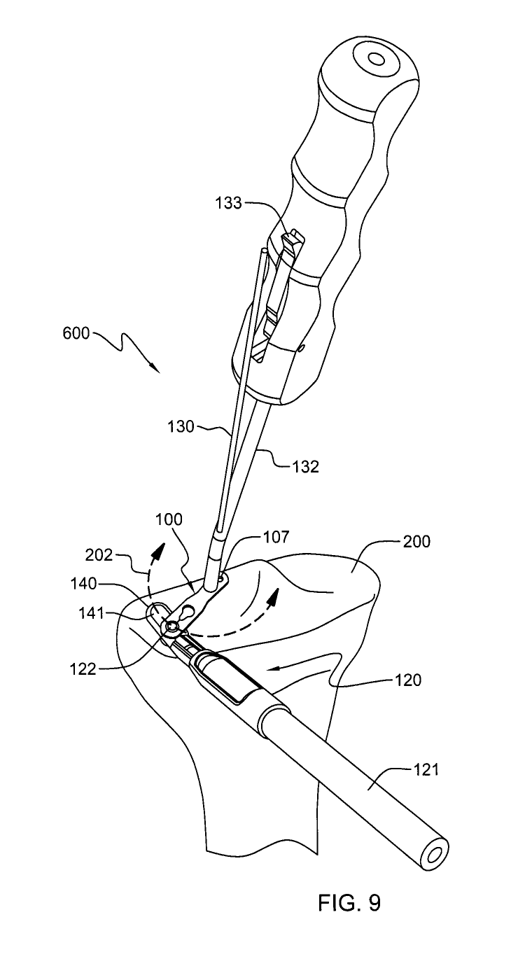

FIG. 9 is a perspective view showing the assembled apparatus 600. FIG. 9 exhibits apparatus 600 in position on the proximal tibia prior to performing an inlayed resection. As discussed above, apparatus 600 may be used to perform an inlay style resection. Inlay resections are performed when the surgeon user desires to maintain a rim of bone around the inner resected bone portions. Some implants are designed to be seated within the inlay resection rather than on top of the resected surface like other implants. The apparatus facilitates the creation of inlay resections, although it may be also used for onlay resections as well when mechanical control of the resection path is needed or the resection location is obstructed so the cutting tool needs to be controlled in some fashion.

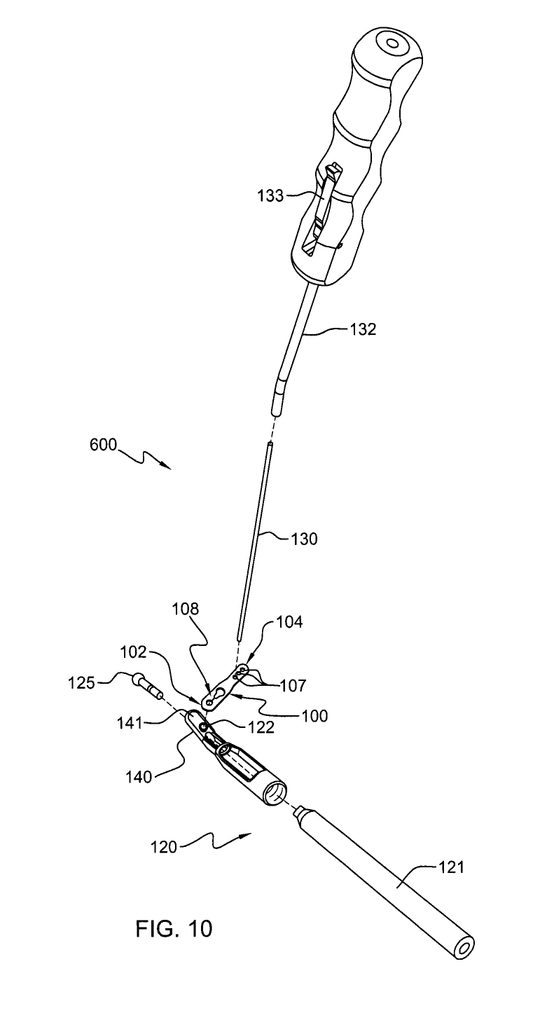

FIGS. 9 and 10 show apparatus 600 to include tether 100 attached to cutting device 120 and reference locator 130 positioned within a bone 200 (only for FIG. 9). Reference locator 130 as shown for example purposes is a wire, although other locators may be uses including k-wires, pins, screws and drill bits. Cutting device 120 further includes bur 124 with cutting head 125 (not seen) extending from a handle 121 with attached bur guard 140. Button 122 is disposed on top surface 141 of guard 140. Positioned over reference locator 130 is a sheath 132 that is tubular so as to allow reference locator 130 to be inserted up into the internal central cavity of sheath 132. Sheath 132 also includes a locking mechanism 133 for securing sheath 132 to reference locator 130. Locking mechanism 133 may be a cam lock or other commercially available locking mechanism

As seen in FIG. 9, apparatus 600 is positioned adjacent to bone 200, with reference locator 130 inserted in the medial aspect of bone 200. Sheath 132 is positioned over sheath 132 to hold tether 100 in close proximity to the proximal surface of bone 200. Tether 100 allows the operator to move cutting device 120 in a controlled fashion along cutting line 202, to cut into bone 200 in an arcuate shape to create an inlayed resection. A rim of cortical bone may remain after cutting device 120 has been moved along cutting line 202. Depending on the size of the implant, the surgeon user will typically start with hole 107 that has the shortest radius and then depending on the size of the proximal tibia will sequentially progress to hole 107 that is positioned closer to medial end 104 results in a large travel radius. The goal being to achieve an intact rim of cortical bone 200 of between 2 and 6 mm in which to seat the inlayed implant.

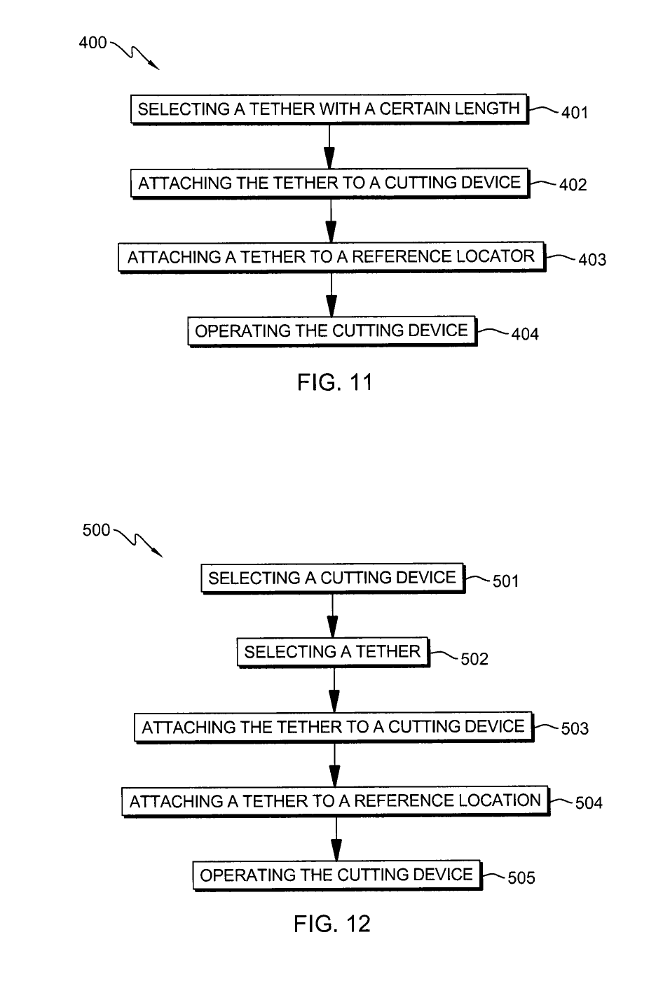

The method of using a tether is to perform a bone resection 400 is generally disclosed herein. It is understood that all possible embodiments of the tether and cutting device previously discussed may be used with the method. Further, for brevity sake, the various structural elements of tether 100 and cutting device 120 disclosed in this method that have been described above, will not be discussed in detail again here and all limitations previously discussed are applicable to the method. As shown in FIG. 11, the method usually includes the step of selecting a tether with a certain length 401. The method may include the further step of attaching the tether to the cutting device 402. An additional step may include attaching the tether to a reference locator 403. It should be understood that the order of steps 402 and 403 may be switched depending on the preference of the surgeon user. The method may further include the step of operating the cutting device with the tether controlling the movement of the cutting device relative to the reference locator during the bone resection. It should be understood that one skilled in the art will utilize standard surgical approaches in method 400.

As illustrated in FIG. 12, a method of creating an inlay bone resection 500 is disclosed. After the surgeon user has created an incision, the method may generally include the step of selecting a cutting device 501. The method may also include the step of selecting a tether which has been adapted to control the movement of the cutting device 502. The method may further include the step of attaching the tether to the cutting device 503. The method may include the step of attaching the tether to the reference locator 504. The method may also have the step of operating the cutting device to create the inlay bone resection 505. It is understood by those skilled in the art that the order of steps 501 and 502 and steps 503 and 504 may to be reversed and will depend on the preference of the surgeon user.

Although the various embodiments have been depicted and described in detail herein, it will be apparent to those skilled in the relevant art that additional modifications, and substitutions can be made without departing from its essence and therefore these are to be considered to be within the scope of the following claims.

What is claimed is:

1. A resection tether for controlling the movement of a cutting device, the resection tether comprising:
an elongated member having a medial end and a lateral end and a longitudinal axis extending there between, the lateral end configured as a means for attaching to a cutting device and the medial end configured as a means for attaching to a reference locator in a bone, wherein the resection tether controls the movement of the cutting device relative to the locator.

2. The resection tether of claim 1, wherein the elongated member further comprises a top surface and a bottom surface.

3. The resection tether of claim 2, wherein the means for attaching to a cutting device comprises at least one opening extending from the top surface to the bottom surface.

4. The resection tether of claim 2, wherein the means for attaching to a cutting device comprises at least two openings extending from the top surface to the bottom surface, the at least two openings are connected by a through slit to facilitate attachment to the cutting device.

5. The resection tether of claim 2, wherein the means for attaching to a reference locator in a bone comprises a plurality of openings extending from the top surface to the bottom surface.

6. The resection tether of claim 5, wherein each of the plurality of openings comprise a center point, the center point for each of the plurality of openings is offset from the longitudinal axis of the elongated member.

7. The resection tether of claim 5, wherein each of the plurality of openings comprise a center point, the center point for each of the plurality of openings is disposed along the longitudinal axis of the elongated member.

8. The resection tether of claim 1, further comprising a handle extending from the medial end.

9. The resection tether of claim 8, wherein the handle is at least one of integral to the medial end and detachable from the medial end.

10. The resection tether of claim 1, wherein the means for attaching to a cutting device and the means for attaching to a reference locator on a bone comprises a loop.

11. The resection tether of claim 1, wherein the means for attaching to a cutting device and the means for attaching to a reference locator on a bone comprises a hook.

12. The resection tether of claim 1, wherein the means for attaching to a cutting device and the means for attaching to a reference locator on a bone comprises a biased clasp.

13. An apparatus for performing a bone resection, comprising:
a tether, the tether comprising an elongated member having a medial end and a lateral end and a longitudinal axis extending there between, the lateral end configured as a means for attaching to a bone cutting device and the medial end configured as a means for attaching to a position on a bone;
a bone cutting device; and
a reference locator positioned within a bone;
wherein the tether connects the bone cutting device to the reference locator to control motion between the bone cutting device and the reference locator when performing a bone resection.

14. The apparatus of claim 13, wherein the bone cutting device comprises:
a cutting bur;
a handle;
a guard connected to the handle having a top surface and a bottom surface; and
wherein the cutting bur is operatively connected to the handle and is positioned adjacent to the bottom surface of the guard when in operation.

15. The apparatus of claim 14, wherein the guard comprises an attachment mechanism disposed on the top surface adapted to couple to the lateral end of the tether.

16. The apparatus of claim 15, wherein the attachment mechanism comprises at least one of a button, a screw, a clip, a clasp, a post and at least one projection.

17. The apparatus of claim 13, further comprises a sheath device adapted to facilitate attachment of the tether to the reference locator.

18. The apparatus of claim 17, wherein the sheath device comprises a tubular body and a locking mechanism, the tubular body is adapted to extend over the reference locator and when the locking mechanism is actuated, moveably secure the medial end of the tether juxtaposed to a bone surface.

19. A method of using a tether to perform a bone resection, the method comprising:
selecting a tether with a certain length;
attaching the tether to a cutting device;
attaching the tether to a reference locator; and
operating the cutting device;
wherein the tether controls the movement of the cutting device relative to the reference locator when performing a bone resection.

20. A method for creating an inlay bone resection, the method comprising:
selecting a cutting device;
selecting a tether adapted to control the movement of the cutting device;
attaching the tether to the cutting device;
attaching the tether to a reference locator; and
operating the cutting device to cut the bone to create an inlay bone resection.
